(19) United States
(12) Patent Application Publication
(10) Pub. No.: US 2021/0071164 A1
(43) Pub. Date: Mar. 11, 2021

(54) METHOD FOR PURIFYING TOTAL MRNA FROM TOTAL RNA USING SLFN13

(71) Applicant: SUN YAT-SEN UNIVERSITY CANCER CENTER (SYSUCC), Guangzhou, Guangdong (CN)

(72) Inventors: Song GAO, Guangzhou (CN); Jinyu YANG, Guangzhou (CN); Wei XIE, Guangzhou (CN); Xiangyu DENG, Guangzhou (CN)

(73) Assignee: Sun Yat-sen University Cancer Center (SYSUCC), Guangzhou, Guangdong (CN)

(21) Appl. No.: 16/625,486

(22) PCT Filed: Apr. 20, 2018
(86) PCT No.: PCT/CN2018/083874

§ 371 Date: Oct. 14, 2020

(30) Foreign Application Priority Data
Mar. 6, 2018 (CN) 201810181007.6

(51) Int. Cl. C12N 15/10; C12Q 1/6806

(52) U.S. Cl. CPC C12N 15/1003; C12Q 2521/301; C12Q 1/6806

(57) ABSTRACT

Provided is a method for purifying total mRNA from total RNA with SLFN13, comprising the following steps of: (1) total RNA extraction; (2) enzyme digestion of tRNA and rRNA in the total RNA by using SLFN13; and (3) after the enzyme digestion is completed, directly heating at 70° C. for 15 min to deactivate the enzyme, to obtain the purified total mRNA.

### FIELD OF THE INVENTION

The present disclosure belongs to the field of biotechnology, and particularly relates to a method for purifying total mRNA from total RNA with SLFN13 (Schlafen13).

### BACKGROUND OF THE INVENTION

Messenger RNAs (mRNAs) are essential macromolecules of all organisms. Transcription of mRNA is an indispensible stage during the expression of a gene. mRNAs pass the genetic information stored in DNAs to the cellular translational machinery to faithfully produce various proteins that carry out various biological functions. Therefore, mRNAs can reflect the transcription and expression information of a specific cell or tissue at a certain functional state, and is closely related to cell property, growth situation and the like. High-throughput sequencing of transcriptome mRNA is a highly efficient method that is widely used at present for research and healthcare. By acquiring complete sequence information of mRNAs within a single run, the high-throughput mRNA sequencing method can analyze comprehensive transcriptome information such as gene expression, single nucleotide polymorphism (SNP), new transcripts, new isomers, splicing sites, specific expression of alleles and rare transcription. The first important step during a sequencing experiment is to extract total RNA of target cells or tissues, and obtain, as much as possible, high-quality total mRNA with good integrity and high purity. The high-quality mRNA preparation is the prerequisite for the efficiency of subsequent full cDNA library construction, which is realized through a reverse transcription process before accurate and reliable sequencing results can be obtained. In the total RNA extract of an organism, however, mRNAs typically take up only 1% to 5%, whereas 75% to 85% are ribosome (r)RNAs and 10% to 16% are transfer (t)RNAs. In addition, mRNA molecules are highly inhomogenous in terms of molecular weight and abundance. Therefore, to purify high-quality mRNA from total RNA while ensuring the integrity is an indispensible but a difficult step for building a cDNA library. At present, there are mainly two approaches to realize the relative purification of mRNA: one utilizes the fact that most mRNA molecules possess poly(A) sequence at their 3′-termini, and designs a poly dT-containing matrix specifically bound to poly(A), so as to separate poly(A)-containing mRNA from total RNA; the other designs a matrix capable of being specifically bound to a conserved region in rRNA, so as to remove as much as possible rRNA which is the most abundant impurity, of total RNA, and to obtain relatively purified mRNA.

RNA is relatively unstable. It is prone to degradation in vitro, especially when exposed to air. The two purification methods above have relatively complicated processes and are difficult to be finished in short time, which greatly increases the risk for RNA degradation. The degradation of RNA can seriously affect the quality of a library, which not only leads to loss of important information, but also introduces many mistakes and errors. More importantly, the purification effect of both the above two methods are not quite ideal. For the first method, it can only ensure 40% to 70% integrity of purified mRNA, which would affect the accuracy of sequencing data and differential display between data sets. For the second method, though the resulted mRNA integrity is better than that of the first method, the final purity of mRNA is much lower. This is because the second method is impotent to remove tRNA, whose amount greatly exceeds mRNA even after rRNA is removed from the total RNA. Therefore, after the final step of the second method, the resulting RNA pool often contains only less than 30% mRNA that is wanted.

Today, the high-throughput RNA sequencing methods are still developing, and the population of the users continues growing. As the integrity and purity of total mRNA extracted from various samples directly determines the quality of the subsequently cDNA library construction, hence the accuracy of the sequencing data, the quality of the mRNA sample becomes a limiting factor that affects the usage, development, and data interpretation of the high-throughput RNA sequencing method. Therefore, it is urgent to develop novel mRNA purification methods (i.e. time-saving, easy-handling, low-costs, and purer mRNA), to avoid the drawbacks of the traditional methods as stated above.

### SUMMARY OF THE INVENTION

The present disclosure is intended to overcome the defects and shortcomings of the methods for purifying total mRNA from total RNA in the prior art above, which have complicated steps, non-ideal purification effect, and difficulty in ensuring quality and integrity of mRNA. The present disclosure provides a good method for purifying total mRNA from total RNA with SLFN13. According to the present disclosure, total mRNA is purified from total RNA by using SLFN13 that specifically digests tRNA and rRNA. The method of the present disclosure not only greatly improves the purity of total mRNA, but also simplifies experimental process, saves time, and ensures the stability and integrity of total mRNA, thereby ensuring the accuracy and effectiveness of subsequent library establishment, sequencing data and other relevant experimental analysis.

Regarding the shortcomings of the two traditional purification methods above, we purify total mRNA by a novel method which introduces a specific endonuclease targeting and digests tRNA and rRNA that have the highest content in total RNA into small molecular fragments, without affecting the integrity of mRNA. This method is simple and convenient, and does not need complicated processes such as binding to a purified matrix and elution, thereby greatly reducing the degradation probability of mRNA and ensuring the purity of mRNA.

The object of the present disclosure is achieved through the following technical solutions: a method for purifying total mRNA from total RNA with SLFN13, which may comprise the following specific steps:

(1) extracting total RNA: extracting complete total RNA from a sample using a traditional TRIzol-chloroform method;

(2) performing enzyme digestion on tRNA and rRNA: taking the purification of 10 μg total RNA as an example, adding 1 μl of 50 μM SLFN13 into 10 μl total RNA of 1 μg/ul, then 2 μl 10× enzyme digestion buffer, and 7 μl ddH₂O to generate a 20 μl enzyme digestion system (that is, 1 μg of total RNA is enzymatically digested with 5 μmol of SLFN13, and the enzyme is provided in a concentration of 50 μM); the 10× enzyme digestion buffer may comprise 400 mM Tris-HCl (pH 8.0), 200 mM KCl, 40 mM MgCl₂ and 20 mM DTT; the enzyme digestion system are incubated for 30 min at a room temperature; it can be demonstrated by FIGS. 2 to 5 that tRNA and rRNA in total RNA are specifically digested with SLFN13-N into fragments within 100 nt; and if there are differences in the components due to different sources of the total RNA, the usage amount and digestion time of the enzyme may be appropriately increased or decreased, which are recommended to fluctuate within a range of 30%.

(3) after the enzyme digestion, directly heating at 70° C. for 15 min to inactivate SLFN13-N, so as to obtain purified total mRNA.

The sample in step (1) may be a cell sample or tissue sample.

If the sample in step (1) is special, the total RNA can be extracted by other effective methods, provided that the quality and integrity of the total RNA can be ensured as far as possible.

The SLFN13 in step (2) is one of full-length SLFN13 or an N-terminal domain of SLFN13.

The N-terminal domain of SLFN13 (collectively called SLFN13-N) is a polypeptide containing the amino acid sequence 1-355 of human SLFN13 (hSLFN13-N) or a polypeptide containing the amino acid sequence 1-353 of rat SLFN13 (rSLFN13-N).

The Gene ID corresponding to human SLFN13 is 146857, and its amino acids 1-355 may be mainly purified for use.

The Gene ID corresponding to rat SLFN13 is 303378, and its amino acids 1-353 may be mainly purified for use.

The N-terminal domain of SLFN13 may be prepared by the following expression and purification methods.

The N-terminal domains of SLFN13 (the amino acid sequence 1-355 of human SLFN13, hSLFN13-N, and the amino acid sequence 1-353 of rat SLFN13, rSLFN13-N, collectively called SLFN13-N) may be individually inserted into a pET28 vector. After verification by Sanger DNA sequencing, the recombinant plasmids with correct insert can be transformed into a Rossetta (DE3) expression strain. Bacterial monocolonies can be applied to 100 ml LB medium co-supplied with kanamycin and chloramphenicol for preculture. After 12 to 16 hours, the bacteria culture may be transferred, in a ratio of 1:100, into 5 L TB medium co-supplied with kanamycin and chloramphenicol to expand at 37° C. When optical density of the bacterial culture reaches 0.4 to 0.6, the bacteria solution may be cooled to 17° C. and added with 80 μM IPTG to induce the expression of SLFN13-N protein. After induced expression at 17° C. for 16 h to 20 h, the bacteria culture may be centrifuged to collect and lyse the bacteria to release proteins. A 6×His-tag at the N-terminal of the SLFN13-N can be used for affinity purification with a Ni-matrix. Finally, homogeneous protein components can be separated by size-exclusion chromatography and concentrated to about 2 μg/μl (50 μM) for later use, and frozen at −80° C. for storage. The purification results are shown in FIG. 1.

As a preferred strategy, if there are stricter requirements for the purity of mRNA, total mRNA with higher purity would be obtained by removing the digested tRNA and rRNA fragments in combination with a corresponding small RNA purification kit, after the enzyme is inactivated in step (3).

The total mRNA prepared according to the present disclosure can be directly used in subsequent library establishment.

The present disclosure has the following advantages as compared to the prior art.

The present disclosure breaks the traditional concepts of RNA purification, and introduces a specific RNA endonuclease to digest and remove tRNA and rRNA from total RNA, which is simple, convenient and highly-efficient. The advantages of present disclosure can be further summarized as follows.

1) Low cost and easy availability. The purification process of the present disclosure does not need too many additional RNA purification media, such as specific RNA binding matrices, special RNA purification buffer solutions and the like. The most critical step is to purify and obtain the active endonuclease, which can be expressed by Escherichia coli strains, and be obtained with purity more than 90% by Ni-matrix affinity chromatography combined with size-exclusion chromatography. About 50 mg of protein (about 12 mM) can be obtained by the purification of 3 L bacteria culture, which can express the endonuclease under induction. In addition, the enzyme has high efficiency of enzyme digestion in vitro, and 4 pmol of the enzyme can digest 1 μg of RNA substrate in 10 to 20 min at room temperature. Therefore, the time for purifying the enzyme is short and the cost is low, however, the enzyme can be used for many times.

2) Simple and convenient operation. The use of RNA purification matrix is omitted, and the matrix balance, RNA specific-binding and elution and other processes are thus skipped. It only needs one step, adding a suitable amount of the enzyme into the RNA enzyme digestion system. In the enzyme digestion process, it just needs standing or simple rotation, without additional manual monitoring. For RNA fragments produced by enzyme digestion, they can omit a purification step, because these fragments with very small sizes do not produce too much interference to the library establishment of mRNA with larger molecular weight. The whole process is easy to be mastered and is not easy to introduce errors, and can be operated quickly and skillfully even by a beginner.

3) Time saving. Since the operation steps and experimental processes are simple, it takes less time, which can shorten the experimental period and help to ensure the stability of the RNA samples. Even if the digested fragments are to be removed, the method can be finished within half an hour in the combination with a small RNA extraction kit.

4) Good purification effect. The tRNA and rRNA can be selectively removed in one step to ensure the purity and integrity of total mRNA. The present disclosure mainly relies on the specific endonuclease to digest and remove unnecessary RNA components. Due to the selectivity and specificity of the endonuclease digestion, tRNA and rRNA with the highest content in total RNA can be digested and removed at one time, which is more thorough than other purification methods. Since the enzyme has no digestion activity for single-stranded RNA, the integrity of mRNA can be ensured as much as possible.

5) Contributing to ensure the stability of mRNA. RNA is easy to be degraded in air. In the method of the present disclosure, it can greatly reduce the degradation probability of mRNA introduced in the experimental operation process, which is beneficial to ensure the quality of purified total mRNA, since the time-consuming steps such as sample loading and elution have been omitted.

### BRIEF DESCRIPTION OF THE DRAWINGS

FIG. 1 is a diagram illustrating the SDS-PAGE analysis results of the samples prepared from the collection tubes corresponding to the elution peaks of monomeric protein, after the purification of hSLFN13-N and rSLFN13-N.

FIG. 2 illustrates the urea-gel electrophoresis analysis results after the selective digestion of tRNA with SLFN13-N.

FIG. 3 illustrates the determination of the active sites of SLFN13-N and the detection results of the digestion activity for mature tRNA in vivo. FIGS. 3a and 3b respectively illustrate the enzyme digestion effects of hSLFN13, rSLFN13 and related mutants thereof for small RNA extracted from 293T cells. FIGS. 3c and 3d respectively illustrate enzyme digestion effects of hSLFN13, rSLFN13 and the related mutants thereof for small RNA extracted from HeLa cells. The small RNA mainly contains tRNA, and 5S and 5.8S rRNA.

FIG. 4 illustrates the Northern blot results for verifying the enzyme digestion activity of SLFN13-N for tRNA and rRNA in total RNA extracted in vivo. FIGS. 4a to 4c respectively illustrate the digestion results of SLFN13 for tRNA^{Ser}, tRNA^{Gly} and tRNA^{Lys}, which are detected by specific probes targeting these three mature tRNA. FIG. 4d illustrates the digestion results of SLFN13 for 5S rRNA, which are detected by a probe targeting 5S rRNA.

FIG. 5 illustrates the digestion results of SLFN13-N for rRNA in the total RNA extracted from the cells. FIG. 5a illustrates the digestion results of SLFN13 (hSLFN13 and rSLFN13) and the related mutants thereof for the total RNA extracted from 293T cells. FIG. 5b illustrates the digestion results of SLFN13 (hSLFN13 and rSLFN13) and the related mutants thereof for the total RNA extracted from HeLa cells.

### DETAILED DESCRIPTION OF THE EMBODIMENTS

Hereinafter, the present disclosure will be further described in details with reference to the embodiments, but the embodiments of the present disclosure are not limited to this.

### EXAMPLE 1

### Expression and Purification of SLFN13

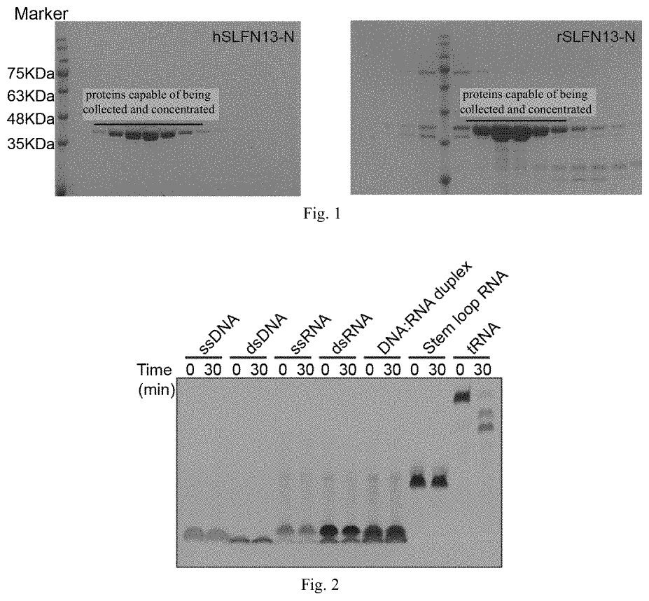

The N-terminal domains of SLFN13 (the amino acids sequence 1-355 of human SLFN13, hSLFN13-N, and the amino acids sequence 1-353 of rat SLFN13, rSLFN13-N, collectively called SLFN13-N) were individually inserted into a pET28 vector. After verification by Sanger DNA sequencing, the recombinant plasmids were transformed into a Rossetta (DE3) expression strain. Bacterial monocolonies can be applied into 100 ml LB medium co-supplied with kanamycin and chloramphenicol for preculture. After 12 to 16 hours, the bacteria culture was transferred, in a ratio of 1:100, into 5 L TB medium co-supplied with kanamycin and chloramphenicol to expand at 37° C. It was cooled to 17° C. when OD reached 0.4 to 0.6, and 80 μM IPTG was added to induce the expression of SLFN13-N protein. After induced expression at 17° C. for 16 h to 20 h, the bacteria culture was centrifuged to collect and lyse the bacteria to release the proteins. Then, a 6×His-tag at the N-terminal of SLFN13-N was used for affinity purification with a Ni-matrix. Finally, the homogeneous protein components were separated by size-exclusion chromatography, concentrated to about 2 ng/μl (50 μM) for later use, and frozen at −80° C. for storage. The purification results of hSLFN13-N and rSLFN13-N are shown in FIG. 1. It can be seen from FIG. 1 that the purity of both the proteins are more than 90%.

### EXAMPLE 2

The present disclosure provides a method for purifying total mRNA from total RNA by using SLFN13, which comprises the following specific steps of:

(1) Extracting total RNA: extracting complete total RNA by using a traditional TRIzol-chloroform method for cell or tissue samples (if the samples are special, other applicable methods can be considered, provided that the quality and integrity of the total RNA can be ensured as far as possible).

(2) Performing enzyme digestion on tRNA and rRNA: calculating the amount of SLFN13 endonuclease to be added and the digestion time, according to the amount of total RNA extracted from the sample, and an approximate content ratio of tRNA, rRNA and mRNA therein. Taking contents of tRNA, rRNA and mRNA in the total RNA being respectively 12%, 83% and 3% as an example, if 10 μl total RNA (1 μg/ul) was taken, 1 μl SLFN13 (50 μM), 2 μl 10× enzyme digestion buffer, and 7 μl ddH₂O were added to obtain 20 μl enzyme digestion system (that is, 1 μg total RNA is enzymatically digested with 5 pmol SLFN13, and the enzyme is provided in a concentration of 50 μM). The 10× enzyme digestion buffer comprised 400 mM Tris-HCl (pH 8.0), 200 mM KCl, 40 mM MgCl₂ and 20 mM DTT. The digestion system was incubated at a room temperature for 30 min. It was demonstrated by FIGS. 2 to 5 that tRNA and rRNA in the total RNA are specifically digested by SLFN13-N to fragments within 100 nt. If there are differences in the components due to the different sources of the total RNA, the usage amount of the enzyme can be appropriately increased or decreased, and is recommended to fluctuate within a range of 30%.

(3) After the enzyme digestion, directly heating at 70° C. for 15 min to inactivate SLFN13-N.

(4) If there were stricter requirements for the purity of mRNA, total mRNA with higher purity was obtained for subsequent library establishment, by removing the digested tRNA and rRNA fragments in combination with a corresponding small RNA purification kit, after the step (3).

FIG. 2 illustrates the selective digestion results of SLFN13-N for tRNA. As shown by the figure, SLFN13-N was incubated with different types of nucleic acid substrates in vitro for enzyme digestion of 30 min and then the urea gel electrophoresis analysis was performed, and the results show that only tRNA is specifically digested by SLFN13-N.

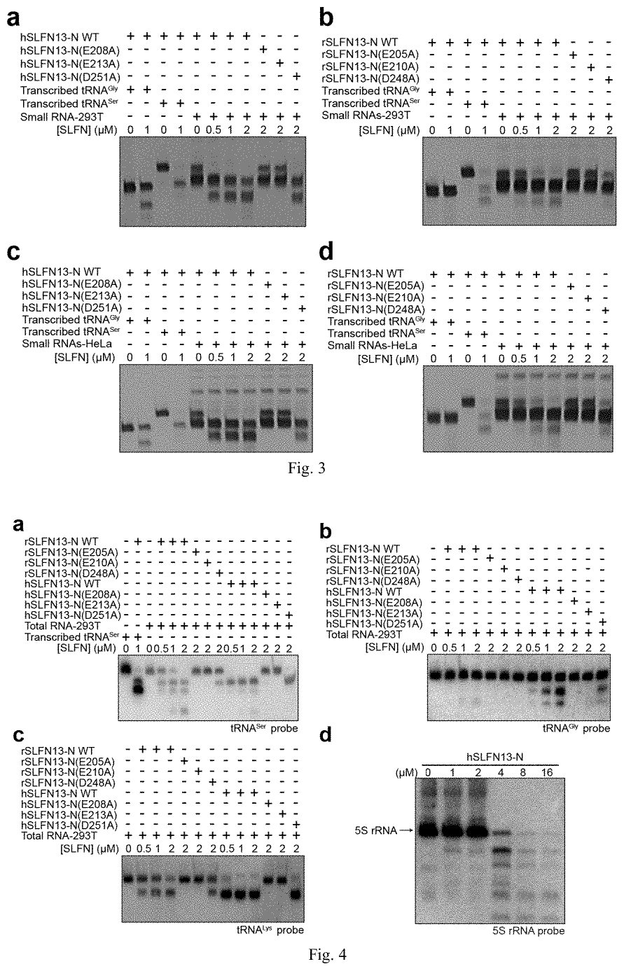

FIG. 3 illustrates the determination of the active sites of SLFN13-N and the digestion activity thereof for mature tRNA in vivo. In order to better understand the digestion property of SLFN13-N, we determined the active sites of the enzyme digestion and verified the digestion activity of related mutant proteins for mature tRNA extracted from cells. We extracted small RNA with more than 90% tRNA content from HEK-293T cells and HeLa cells as substrates for the verification of enzyme digestion. The results show that SLFN13-N has similar digestion activity and property for tRNA extracted from the cells and tRNA transcribed in vitro.

FIG. 4 illustrates the Northern blotting results for verifying the enzyme digestion activity of SLFN13-N for tRNA and rRNA in the total RNA extracted in vivo. The probes targeting tRNA^{Ser}, tRNA^{Gly}, tRNA^{Lys} and 5S rRNA were designed respectively, and were labeled with P32 at 5′ end thereof. The total RNA extracted from the cells was incubated and reacted with SLFN13-N, and then separated on a gel and transferred to a membrane. The corresponding probes were respectively hybridized with the products of the enzyme digestion. The results show that SLFN13-N has obvious enzyme digestion activity for tRNA and 5S rRNA in the total RNA extracted from the cells, and with the increase of the time and the amount of the enzyme, 5S rRNA can be enzymatically digested into fragments.

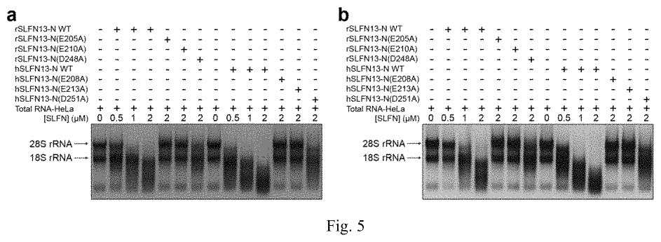

FIG. 5 illustrates the enzyme digestion activity of SLFN13-N for rRNA in the total RNA extracted from the cells. SLFN13-N and related enzymatically active mutants were respectively incubated and reacted with the total RNA extracted from HEK-293T cells and HeLa cells. With the increase of the concentration of the enzyme, the digestion effect was significantly enhanced, and with the increase of the time, rRNA was gradually digested into fragments.

The present disclosure breaks the traditional concepts of RNA purification, and introduces a specific RNA endonuclease to digest and remove tRNA and rRNA molecules from the total RNA, which is simple, convenient and highly-efficient. The advantages of the present disclosure can be summarized as follows.

1) Low cost and easy availability. The purification process of the present disclosure do not need too many additional RNA purification media, such as specific RNA binding matrices, special RNA purification buffer solutions and the like. The most critical step is to purify and obtain the active endonuclease, which can be expressed by Escherichia coli strains, and be obtained with a purity more than 90% by Ni-matrix affinity chromatography combined with size-exclusion chromatography. About 50 mg of proteins (about 12 mM) can be obtained by the purification of 3 L bacteria culture, which can express the endonuclease under induction. In addition, the enzyme has high efficiency of enzyme digestion in vitro, and 4 pmol of the enzyme can digest 1 μg of RNA substrate in 10 to 20 min at room temperature. Therefore, the time for purifying the enzyme once is short and the cost is low, however, the enzyme can be used for many times.

2) Simple and convenient operation. The use of RNA purification matrix is omitted, and the matrix balance, RNA specific-binding and elution and other processes are thus skipped. It only needs one step, adding a suitable amount of the enzyme into the RNA enzyme digestion system. In the enzyme digestion process, it just needs standing or simple rotation, without additional manual monitoring. For RNA fragments produced by enzyme digestion, they can omit a purification step, because these fragments with very small sizes do not produce too much interference to the library establishment of mRNA with larger molecular weight. The whole process is easy to be mastered and is not easy to introduce errors, and can be operated quickly and skillfully even by a beginner.

3) Time saving. Since the operation steps and experimental processes are simple, it takes less time, which can shorten the experimental period and help to ensure the stability of the RNA samples. Even if the digested fragments are to be removed, the method can be finished within half an hour in the combination with a small RNA extraction kit.

4) Good purification effect. The tRNA and rRNA can be selectively removed in one step to ensure the purity and integrity of total mRNA. The present disclosure mainly relies on the specific endonuclease to digest and remove unnecessary RNA components. Due to the selectivity and specificity of the endonuclease digestion, tRNA and rRNA with the highest content in the total RNA can be digested and removed at one time, which is more thorough than other purification methods. Since the enzyme has no enzyme digestion activity for single-stranded RNA, the integrity of mRNA can be ensured as much as possible.

5) Contributing to ensure the stability of mRNA. RNA is easy to be degraded in air. In the method of the present disclosure, it can greatly reduce the degradation probability of mRNA introduced in the experimental operation process, which is beneficial to ensure the quality of purified total mRNA, since the time-consuming steps such as sample loading and elution have been omitted.

The embodiments above are preferred embodiments of the present disclosure, but not intended to limit the embodiments of the present disclosure. Any amendment, modification, replacement, combination and simplification can be made, without deviating from the spiritual substance and principle of the present disclosure, and shall be equivalent substitute modes and all fall within the protection scope of the present disclosure.

1. A method of purifying total mRNA from total RNA with SLFN13, comprising the following steps:
(1) extracting complete total RNA from a sample by using a traditional TRIzol-chloroform method;
(2) performing enzyme digestion of tRNA and rRNA: taking purification of 10 μg total RNA as an example, adding 1 μl of 50 μM SLFN13 into 10 μl total RNA of 1μg/μl concentration, adding 2 μl 10× enzyme digestion buffer, and adding 7 μl of ddH₂O to obtain 20 μl of an enzyme digestion system, wherein of the digestion buffer comprises 400 mM of Tris-HCl (pH 8.0), 200 mM of KCl, 40 mM of MGC L2 and 20 mM of DTT; and incubating the enzyme digestion system at room temperature for 30 min; and
(3) after the enzyme digestion, heating the enzyme digestion system at 70° C. for 15 min to deactivate the SLFN13-N to obtain purified total mRNA.

2. The method of claim 1, wherein the sample in step (1) is one of a cell sample or a tissue sample.

3. The method of claim 1, wherein the total RNA can be extracted by other effective methods if the sample in step (1) is special.

4. The method of claim 1, wherein the SLFN13 in step (2) is one of a full-length SLFN13 or an N-terminal structural domain of SLFN13.

5. The method of claim 4, wherein the N-terminal structural domain of SLFN13 is one of an amino acid sequence 1-355 of human SLFN13 or an amino acid sequence 1-353 of rat SLFN13.

6. The method of claim 5, wherein the human SLFN13 has a Gene ID of 146857, of which the amino acid sequence 1-355 is mainly purified for use.

7. The method of claim 5, wherein the rat SLFN13 has a Gene ID of 303378, of which the amino acid sequence 1-353 is mainly purified for use.

8. The method of claim 4, wherein the N-terminal structural domain of SLFN13 is prepared by the following expression and purification method:
constructing the N-terminal structural domain of SLFN13 into a pET28 vector;
after verifying the N-terminal structural domain through sequencing, transforming plasmids into a Rossetta (DE3) expression strain;
selecting monocolonies to preculture in 100 ml of LB medium added with double-antibiotics, kanamycin and ampicillin;
after 12 to 16 h, transferring a bacteria solution in a ratio of 1:100 into 5 L of TB medium added with the double-antibiotics to expand at 37° C.;
cooling to 17° C. when OD reaches 0.4 to 0.6;
adding 80 μM of IPTG to induce expression of SLFN13-N protein;
after induced expression at low-temperature for 16 to 20 h, centrifuging the bacteria solution to collect and break the bacteria to release proteins; and
finally separating a homogeneous protein component by size-exclusion chromatography, and concentrating to about 2 μg/μl for later use, and freezing at −80° C. for storage
wherein the N-terminal of SLFN13-N comprises a 6×His-tag, which can be performed affinity purification with a Ni-matrix.

9. The method of claim 1, if there are stricter requirements for purity of the mRNA, after the enzyme is deactivated in step (3), further comprising removing the digested tRNA and rRNA fragments in combination with a corresponding small RNA purification kit to obtain the total mRNA with a higher purity.


SEQUENCE LISTING (4 sequences)

SEQ ID NO: 1 - DNA, 2694 bp, Homo sapiens
atggaggcaaatcactgctccctgggtgtgtatccatcttacccagacctggtcatcgatgtcggagaagtgactctgggagaagaaaacagaaaaaagctacagaaaactcagagagaccaagagagggcgagagttatacgggccgcgtgtgctttattaaactcaggaggaggagtgattcagatggaaatggccaacagggatgagcgtcccacagagatgggactggatttagaagaatccttgagaaagcttattcagtatccatatttgcaggctttctttgagactaagcaacacggaaggtgtttttatatttttgttaaatcttggagtggtgatcctttccttaaagatggttctttcaattcccgcatttgcagccttagttcttcattatactgtagatctggcacctctgtgcttcacatgaattcaagacaggcattcgatttcctgaagaccaaggaaagacagtccaaatataatctgattaatgaagggtctccacctagtaaaattatgaaagctgtataccagaacatatctgagtcaaatcctgcatatgaagttttccaaactgacactattgaatatggtgaaatcctatcttttcctgagtctccatccatagagtttaaacagttctctacaaaacatatccaacaatatgtagaaaatataattccagagtacatctctgcatttgcaaacactgagggaggctatctttttattggagtggatgataagagtaggaaagtcctgggatgtgccaaagaacaggttgaccctgactctttgaaaaatgtaattgcaagagcaatttctaagttgcccattgttcatttttgctcttcaaaacctcgggtagagtacagcaccaaaatcgtagaagtgttttgtgggaaagagttgtatggctatctctgtgtgattaaagtgaaggcattctgttgtgtggtgttctcggaagctcccaagtcatggatggtgagggagaagtacatccgccccttgacaactgaggaatgggtagagaaaatgatggacgcagatccagagtttcctccagactttgctgaggcctttgagtctcagttgagtctatctgacagtccttcactttgcagaccagtgtattctaagaaaggtctggaacacaaagctgatctacaacaacatttatttccagttccaccaggacatttggaatgtactccagagtccctctggaaggagctgtctttacagcatgaaggactaaaggagttaatacacaagcaaatgcgacctttctcccagggaattgtgatcctctctagaagctgggctgtggacctgaacttgcaggagaagccaggagtcatctgtgatgctctgctgatagcacagaacagcacccccattctctacaccattctcagggagcaggatgcagagggccaggactactgcactcgcaccgcctttactttgaagcagaagctagtgaacatggggggctacaccgggaaggtgtgtgtcagggccaaggtcctctgcctgagtcctgagagcagcgcagaggccttggaggctgcagtgtctccgatggattaccctgcgtcctatagccttgcaggcacccagcacatggaagccctgctgcagtccctcgtgattgtcttactcggcttcaggtctctcttgagtgaccagctcggctgtgaggttttaaatctgctcacagcccagcagtatgagatattctccagaagcctccgcaagaacagagagttgtttgtccacggcttacctggctcagggaagaccatcatggccatgaagatcatggagaagatcaggaatgtgtttcactgtgaggcacacagaattctctacgtttgtgaaaaccagcctctgaggaactttatcagtgttagaaatatctgccgagcagagacccggaaaactttcctaagagaaaaatttgaacacattcaacacatcgtcattgacgaagctcagaatttccgtactgaagatggggactggtataggaaggcaaaaaccatcactcagagagaaaaggattgtccaggagttctctggatctttctggactactttcagaccagtcacttgggtcacagtggccttccccctctctcagcacagtatccaagagaagagctcaccagagtagttcgcaatgcagatgaaatagccgagtacatacaacaagaaatgcaactaattatagaaaatcctccaattaatatcccccatgggtatctggcaattctcagtgaagctaaatgggttccaggtgttccaggcaacacaaagattattaaaaactttactttggagcaaatagtgacctatgtggcagacacctgcaggtgcttctttgaaaggggctattctccaaaggatgttgctgtgcttgtcagcaccgtgacagaagtggagcagtatcagtctaagctcttgaaagcaatgaggaagaaaatggtggtgcagctcagtgatgcatgtgatatgttgggtgtgcacattgtgttggacagtgtccggcgattctcaggcctggaaaggagcatagtgtttgggatccatccaaggacagctgacccagctatcttacccaatattctgatctgtctggcttccagggcaaaacagcacctatatatttttctgtga

SEQ ID NO: 2 - protein, 897 aa, Homo sapiens [sequence omitted]

SEQ ID NO: 3 - DNA, 2724 bp, Rattus norvegicus
atggaaattcatccctccttagtggtggaaccgtcttacccagacttgatcatccatgcaggagaggtgacactcggagaaaaagatcgaaataaaatggactcgaagaagaagagactggagaaagcaagaatcacagaggctgcttgtgctctgttgaactctggaggaggagtgattgttatgcaaatgtccaacaagagcgagcatcctgtggagatgggactggacttggagacgtctttaagagagcttattccatcctctgatttgcaggctttcattgagaccaagcaacaaggggacctgttttacatttttgttaaatcttggagctgtagccctaaagatggttccactaagcctcgaatttgcagcctgagctcttccctgtactgtcgatctttaacttcaaagcttcccctggattcgaaagaaacgtttgagtttctggagcgcaagaagacgtgtgtcaaaggtagtctgactgacggcaaaggtccacctgctaaaattcccagactcatgtatcagaacgaccttgaatcaaatcccgcttttgaaattttccaaagtgagagactcgaatatggccagcgcttgccgttttctgaatctgcatctatcgagtttaaacaattctctaccagacgtgcccacgaatatataaaaagtgtaattccagagtacatttccgcatttgcaaacacccagggaggctatcttttatttggggtggatgatgagagtaagagagtcctgggatgcccaaaagacaatgttgaccgcgactctttgaaagcagtggtaaatgaagcaatatccaagttgccagttttccatttttgttcatctaaagagaaggtgtcttataagaccagagtcatagatgtgtttaaagaaggaaatttgtatggttatctctgtgtgatcaaagtagagaggttctgctgtgcagtattctcagaggctcccatttcatggatggcagacaaggagaacggtgtctacagcctgaacactgagaaatgggttcgcatgatggtggatattggcccagaggcagcttccagcaagcaatcctccctcgatgacctgtctaaagattttgaatgtcagctgagtctgtccaacagccctccacactgcagaccagtgtattctaaaaaaggactggaacataaaggggacctgcaaaagcgtttatttcaagtgtcagcagactgttttaagtatactcctgaatccctctggagagagctgtgttcccagcatgagagactagagaacttaataagccagcaaatgtgttctttctcctgtggtttgctgatcctctctagaagctgggctgtggacctgaacctggaagggaagcagggagtcatctgtgatgctctgctcattgcagagaatagccccccgacactctacaccatccttgaggagcaagatgagctaggtcaggactactgcacccgcactgcctttactctgaagcagaagctggtgaacactggtggctacactgggagagtgtgtgtcatgaccaaggttctctgcctgagttctcagaacaatattgagaccaatgggaactcagtttctcttattgattacccacgctcctataaccttgcaaatatccaggaaatggaggacttgctgcaggcccttgtgattgtcttgctcaacttcagctctttcttgagcgaccagcttggctgtgaaattttgaatcttctcacagttcaacagtatgagatactctcaaaaagtctccacaaaaccagaaaactgtttgtgcatggcatgccaggctcagggaagaccatcattgccatgaaaatcatggaaaagatcaaaaacacattccactgtgaaagagatagcattctctacatctgtgaaaatcagctgttgagggacttcatccgggcaaaaaatgtctgccgggcagtaacccggaaaacctttatgacacctaactttgaggtggagaagatccaacacattattgttgatgaagcccagaatttccgcactgagaatggggactggtatatgaaggcaaagcgtatcactcagagaatgaaaacttgtcctcaaattttctggatctttctggactattttcagaccagtcaccagaaggagagtggcctcccagatttcttacaccagtatccaaaggaagagctcacaaaagtggtacgcaatgcagataaaatagctgaatttctacaaaaaatatcagagaaaatcagaagtaaccctccacccatcatcccccgagagtccctgaacatggtctgtgagtttaactggtctcaaggtgtatcaggcacctgtaagctgcttacatccttgggtttggaacagatggcgaggtatgtagcagagaggtgttattttttcttgaaaaatggctattccgcccaagacattgcagtgcttttcagtacagaggatgagaaggataacaatgaagatatgttcctgagagaaataaggaacagaacatctcagatagatgatgcctatcatctttacatgtttgacagcatacgtcgattctcgggcctagaaagaagcattgtgtttggtattgatccccgtacagctgagaagtccattttccacaacctaatgctctgcctagcttccagagcgaggaaacatctgtatatcctatctaaagtacccaaccctttcaacttctga

SEQ ID NO: 4 - protein, 907 aa, Rattus norvegicus
MEIHPSLVVEPSYPDLIIHAGEVTLGEKDRNKMDSKKKRLEKARITEAACALLNSGGGVIVMQMSNKSEHPVEMGLDLETSLRELIPSSDLQAFIETKQQGDLFYIFVKSWSCSPKDGSTKPRICSLSSSLYCRSLTSKLPLDSKETFEFLERKKTCVKGSLTDGKGPPAKIPRLMYQNDLESNPAFEIFQSERLEYGQRLPFSESASIEFKQFSTRRAHEYIKSVIPEYISAFANTQGGYLLFGVDDESKRVLGCPKDNVDRDSLKAVVNEAISKLPVFHFCSSKEKVSYKTRVIDVFKEGNLYGYLCVIKVERFCCAVFSEAPISWMADKENGVYSLNTEKWVRMMVDIGPEAASSKQSSLDDLSKDFECQLSLSNSPPHCRPVYSKKGLEHKGDLQKRLFQVSADCFKYTPESLWRELCSQHERLENLISQQMCSFSCGLLILSRSWAVDLNLEGKQGVICDALLIAENSPPTLYTILEEQDELGQDYCTRTAFTLKQKLVNTGGYTGRVCVMTKVLCLSSQNNIETNGNSVSLIDYPRSYNLANIQEMEDLLQALVIVLLNFSSFLSDQLGCEILNLLTVQQYEILSKSLHKTRKLFVHGMPGSGKTIIAMKIMEKIKNTFHCERDSILYICENQLLRDFIRAKNVCRAVTRKTFMTPNFEVEKIQHIIVDEAQNFRTENGDWYMKAKRITQRMKTCPQIFWIFLDYFQTSHQKESGLPDFLHQYPKEELTKVVRNADKIAEFLQKISEKIRSNPPPIIPRESLNMVCEFNWSQGVSGTCKLLTSLGLEQMARYVAERCYFFLKNGYSAQDIAVLFSTEDEKDNNEDMFLREIRNRTSQIDDAYHLYMFDSIRRFSGLERSIVFGIDPRTAEKSIFHNLMLCLASRARKHLYILSKVPNPFNF